(19) United States
(12) Patent Application Publication
(10) Pub. No.: US 2020/0064255 A1
(43) Pub. Date: Feb. 27, 2020

(54) DEVICE AND METHOD FOR DETERMINING AT LEAST ONE PHYSICAL PARAMETER OF PHARMACEUTICAL PRODUCTS

(71) Applicant: Sotax AG, Thun (CH)

(72) Inventor: Thomas BOSS, Gwatt (CH)

(21) Appl. No.: 16/489,059

(22) PCT Filed: Feb. 22, 2018
(86) PCT No.: PCT/IB2018/051088

§ 371 Date: Aug. 27, 2019

(30) Foreign Application Priority Data
Feb. 28, 2017 (EP) 17158518.5

(51) Int. Cl. G01N 21/13; G01N 35/00; G01N 33/15

(52) U.S. Cl. CPC G01N 21/13; G01N 2021/0339; G01N 33/15; G01N 35/0099

(57) ABSTRACT

The invention relates to a device for determining at least one physical parameter of pharmaceutical products such as, for example, tablets (T), pills, oblongs or similar, the device having at least one examining station (1). The device has, in addition, at least one automatic system for transporting the products to and/or between and/or inside and/or from the or each examining station (1). The transport system has at least one robotic arm (2) having at least one controllable holding mechanism (3) for the pharmaceutical products. The travel paths and the spatial orientation of the holding mechanism (3) can be freely programmed.

### CROSS-REFERENCE TO RELATED APPLICATIONS

The present patent application is a National stage application from PCT application PCT/IB2018/051088 filed Feb. 22, 2018, which claims priority to European patent application EP17158518.5 filed Feb. 28, 2017, all of which incorporated herein by reference by their entirety.

### FIELD OF INVENTION

The invention relates to a device for determining at least one physical parameter of pharmaceutical products such as, for example, tablets, pills, oblongs or similar according to the introductory section of claim 1, as well as to a method for determining at least one physical parameter of pharmaceutical products such as, for example, tablets, pills, oblongs or similar according to the introductory section of claim 16.

### BACKGROUND

In the manufacturing of tablet-shaped pharmaceutical, chemical or food products, such as oblongs, tablets, pills or coated tablets, a number of properties of the products must be borne in mind and complied with. Therefore, for monitoring purposes test specimens are randomly taken from on-going production, collected and examined, measured and if necessary recorded in terms of, among other things, their physical properties, for example, weight, hardness as well as breakdown time in a medium, as well in terms of the dimensions of the sample.

The documents US2014145082, US2015310454, US2016109385, GB2524130, US2010328669 and WO2005074553 describe methods of examining tablets in a predominantly light optical manner. DE102008035830 discloses measurement of tablets as well as a hardness test.

Through WO010744 a device for the automatic quality control of tablet-shaped test specimens has become known. The device comprises a feed-in device which separates the collected test specimens, and a transport star wheel with peripherally arranged receiving chambers for one sample each into which the specimens are fed. Test specimens placed in the receiving chambers are taken step by step to one or more examining stations and, if destruction-free tests are involved, are then ejected into a linear conveyor and collected for archiving purposes. In the case of a destructive test of the specimens ejection of the destroyed specimen usually takes place directly at the relevant station, for example through an opening in a support, forming the lower boundary of the receiving chambers, on which the transporting star rotates. The examination stations are, for example, a hardness tester, weighing scales as well as a device for determining the dimensions, such as length and/or width and/or thickness of the test specimens.

To determine the active substance of the test specimens it is known to carry out a contact-free measurement in which the test specimens are individually scanned by electromagnetic waves of suitable wavelength. For this, NIR testing (NIR: Near Infra-Red) has proven to be particularly expedient, wherein electromagnetic waves in the near infra-red range are used to scan the test specimens.

For example, a system is known which comprises an automatic tablet testing device (for example, for weight, thickness, diameter and hardness) and an NIR examination system. After measurement of its weight and thickness the tablet is discharged and vibrated into a tablet holder. This tablet holder is taken to the NIR system, the measurement is started and the tablet is then deposited in a tube or repository.

A drawback of this method is, for example, that the tablet nests for the different tablet formats are expensive and very awkward to manufacture. So that the tablet can vibrate into it, the holder is made slightly larger than the actual tablet. This results in the fact that undesirable scattered light (light that has not passed through the tablet) may possibly falsify the measuring result.

After pressing the tablets are partially still warm and therefore slightly larger than in the cooled state. Detection (whether the tablet is actually in the holder and 100% orientated) is complex and not reliable. After measurement, e.g. with NIR, the tablets are individually deposited in tablet repository, for subsequent control measurements for example. For this the tablet is blown out of the holder with a current of air and deposited in the relevant tube via an orientation channel. The parts required for this are tablet-specifically manufactured. Disadvantageous here is the process reliability (monitoring with sensors very laborious).

Patent EP2160599 describes the positioning and orientation of an object over a support surface of a pusher. Disadvantageous here is that fact that in the case of concave tablets for example the underside of the pusher has to tablet-specifically adapted. Prevention of the undesirable scattered light is also not resolved 100%. The pusher used here can be a disadvantage if it is desired to bring the tablet to a precisely defined position or record it there or place it in a test tube directly after measurement for example.

To obtain a verifiable measure for quality control, in the case of non-discoid, but, for example, drop-shaped, olive-shaped, cylindrical or torpedo-shaped test specimens, as well as test specimens with a cambered shape it is necessary that the scanning always takes place at the same point or along the same axis of the test specimens.

The aim of the present invention is therefore to create a device and set out a method which do not have the aforementioned disadvantages and, in particular, with low outlay allow precise positioning and possibly also alignment of an object, e.g. a pharmaceutical product to be tested by way of a test apparatus, on a substrate or support surface.

The task is solved by the features of independent claims 1 and 16. Advantageous further embodiments are set out in the figures and in the dependent patent claims.

### SUMMARY OF THE INVENTION

The starting point is a device for determining at least one physical and/or chemical parameter of pharmaceutical products, with at least one examining station as well as with at last one automatic transport system for the products to and/or between and/or within and/or from the or every examining station. The products can, for example, be tablets, pills, oblongs or similar.

In accordance with the invention such a device is characterised in that the transport system comprises a controllable holding mechanism for the form-fitting and/or friction-fitting picking up of the pharmaceutical products, wherein the travel paths and spatial orientation of the holding mechanism are freely programmable. In this way a precisely defined connection of the product with the transport system is ensured, through which in the subsequent test procedures undergone by the product the precise position and spatial location of the product is known and can be adjusted at all times. Particularly flexible handling is therefore possible and incorrect measurements through positioning errors can be minimised.

According to a preferred form of embodiment the holding mechanism is arranged on a preferably freely programmable robotic arm. This tried and tested technology easily allows any position to be travelled to with the pharmaceutical product and also its alignment in any desired spatial position.

According to the invention it is preferably envisaged that at least one of the examining stations is a device for a scanning or reflection measurement. Preferably this device comprises a support or contact surface for the pharmaceutical product.

One advantageous form of embodiment of the device according to the invention is characterised in that the device for scanning or reflection measurement has a support, that is impermeable for the wavelengths used and at least during the scanning measurement partially holds the product in form-fitting manner, and in which a through opening is produced that can be completely covered by the product. In this way measuring errors through extraneous and scattered light can be reduced.

In order to reduce falsification of the measurement through scattered light even further, the support is advantageously made of an elastically flexible material. The pharmaceutical product can be pressed into this material in a form-fitting manner as due to the form and/or friction-fitting holding of the product the transport system can exert corresponding forces on the product.

Preferably, if the holding mechanism of the robotic arm comprises at least two gripper jaws that are adjustable relative to each other, the device according to the invention can be simply and quickly adapted to different pharmaceutical products, controlling for picking up the product is very simple and the products can be very flexibly picked up, transported, positioned and set down.

An advantageous form of embodiment envisages that the gripper surface facing the respective opposite gripper jaw is shaped in such a way that between the gripper jaws an at least two-point support for the product is formed. Through this, good friction-fitting picking up of the products can be brought about by the transport system.

A particularly good holding effect for secure transporting of the pharmaceutical product can be assured by an alternative form of embodiment in which the gripper surfaces of the gripper jaws are shaped in a manner that complements at least part of the peripheral contour of the products.

An embodiment of the invention which is advantageous in that it is simple in design and can be constructed smaller in size is characterised in that the gripper jaws are opposite each other in the form of pincers and are closable when moving towards each other and openable when moving apart.

Alternatively a form of embodiment is also possible in which gripper jaws are arranged along a closed contour and have a central free space for receiving the pharmaceutical product. This variant has the advantage of the best possible picking up and holding effect by way of the transport system.

Particularly advantageous in all gripper variants and forms of embodiment of the transport system is if the controllable holding device and/or any gripper jaws has/have sensors for determining the path of any gripper jaws and/the force exerted on the pharmaceutical products. By way of such a system checking of the spatial orientation and position of the product can already be carried out at the time of picking up the product by the transport system in that the determined values for force and/or path of the gripper jaws, for example, can be compared with reference values for the correct position. This subsequently prevents incorrect measurements due to incorrect positioning of the product.

A further advantageous form of embodiment of the device according to the invention envisages that at the transfer position of the pharmaceutical product to the transport system a turntable is arranged on which the product comes to lie immediately before form-fitting or friction-fitting picking up by the transport system. Thus, in the event of detecting an incorrectly orientated pharmaceutical product (e.g. drop-shaped tablet incorrectly orientated for complementarily shaped grippers), via gripper sensors, optical detection systems or similar, the product can be turned by the turntable into the correct system to be taken up by the transport system.

The holding effect, while simultaneously protecting the products against damage during handling, can be improved further if, in accordance with another optional feature of the invention, the gripping surface of at least one gripper jaw is covered with an elastic layer. Additionally or alternatively the layer lining the gripper jaws can be made of a material that is impermeable to the wavelengths used during the scanning measurement in order to thereby largely prevent incorrect measurements due to scattered as well as extraneous light. This solution is also particularly advantageous for scanning or reflection measurements with tablet holders without an elastic cover layer.

An advantageous form of embodiment of the device according to the invention is characterised in that the gripper jaws have protrusions projecting inwards over a clamped product and that the height of the gripper projecting beyond the base of the protrusion is smaller than the thickness of the products. In this way the holding effect and the prevention of scattered light can be improved even further in the case of scanning measurements.

Particularly preferred is a variant of the invention in which the transport system is configured to exert a pressing force which acts on the product in the direction of the holder.

The solution of the task set out in the introduction is also possible for a method of determining at least one physical parameter of pharmaceutical products, such as, for example, tablets, pills, oblongs or similar. In this, at least one chemical or physical property is checked and the product is transported and manipulated by an automatic transport system between and/or during and/or after at least one checking procedure.

According to the invention, for the solution of the task it is envisaged that the transport path of the product and its spatial orientation is pre-programmed at least partially independently of the spatial technical set-up and is preferably travelled by way of a robotic arm.

Preferably at least one property of the product is also determined by means of a scanning or reflection measurement.

According to an advantageous variant of the method it is envisaged that at least for the scanning measurement the product is fixed in place in relation to the device for scanning measurement, preferably with a pressing force being exerted in the direction of a support.

Alternatively or additionally it can also be envisaged that during the scanning or reflection measurement the product is left moveable. If need be several measurements can also be carried out, preferably at different points of the product. Thus, in the case of elongated products, for example oblongs, a scanning or reflection measurements could take place in the middle and in both end sections in order, for example, to determine a mean value.

Advantageously during at least part of the transport path the product is coupled to the transport system, preferably a holding mechanism of the transporting system in a friction-fitting and/or form-fitting manner.

A further variant of the method according to the invention is characterised in that after completion of all tests the product is placed in a repository or transferred to further testing systems, repositories or manipulation systems by the transport system.

Further advantages, features and details of the invention are set out in the following description in which examples of embodiment of the invention are described with reference to the drawings.

### BRIEF DESCRIPTION OF THE DRAWINGS

The list of reference numbers, as well as the technical content of the patent claims and figures, forms part of the disclosure. The figures are described in an interrelated and overall manner. The same reference numbers denote identical components, reference numbers with different indices denote functionally-identically or similar components.

In these:

FIG. 1 shows a section of a first form of embodiment of a device according to the invention,

FIG. 2 shows a perspective view of a holding mechanism embodied according to the invention,

FIG. 3 shows a longitudinal section through a holding mechanism according to claim 2, placed on a scanning measurement tablet nest,

FIG. 4 shows a perspective view of an alternative embodiment of a holding mechanism,

FIG. 5 shows a perspective view of a further section of a form of embodiment of the device according to the invention,

FIG. 6 shows a perspective view of a gripper jaw device with a closed contour, and

FIG. 7 also shows a schematic view of a further gripper jaw device with a closed contour.

### DETAILED DESCRIPTION OF THE PREFERRED EMBODIMENTS

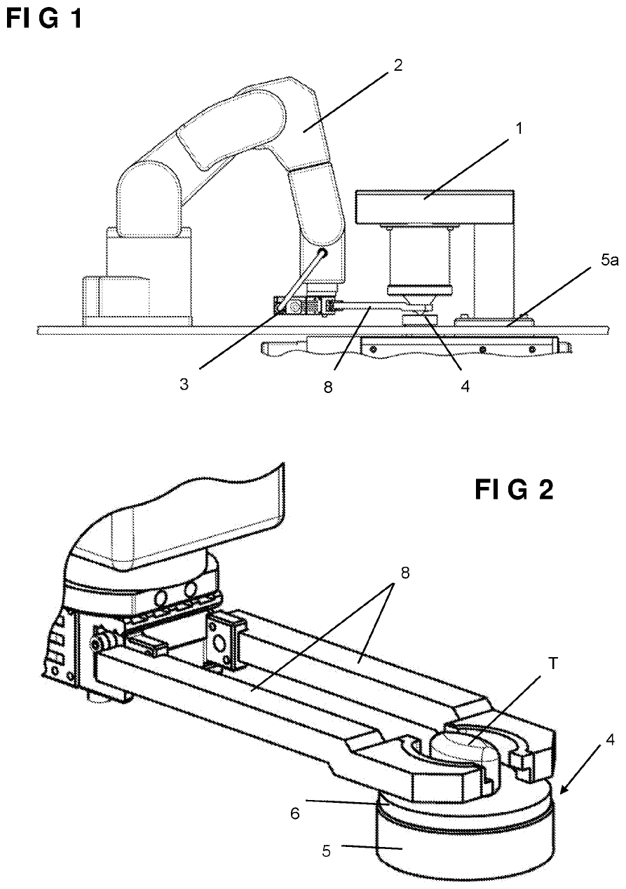

FIG. 1 shows a section of a device for determining at least one physical parameter of pharmaceutical products. These can be tablets, pills, oblongs or similar. The device comprises at least one examining station 1 as well as a least one automatic transport system for the products to and/or between and/or within and/or from the or every examining station 1. The determination of at least one physical parameter of these pharmaceutical products takes place. At least one chemical or physical property is examined, wherein between and/or during and/or after the at least one examination procedure the product is transported and manipulated by an automatic transport system.

In accordance with the invention the transport system comprises at least one robotic arm 2 with at least one controllable holding mechanism 3 for the pharmaceutical products. Preferably the travel paths and the spatial orientation of the holding mechanism 3 on this robotic arm 2 are freely programmable. In this way the pharmaceutical product can also be moved to any position within the reach or the robotic arm 2, and, with appropriate movable attachment of the holding mechanism 3 on the free end of the robotic arm 2, also into any spatial orientation. More particularly, the robotic arm 2 can be controlled in such a way that the transport path of the product and its spatial orientation are pre-programmed at least partially independently of the spatial technical set-up and travelled by way of the robotic arm 2.

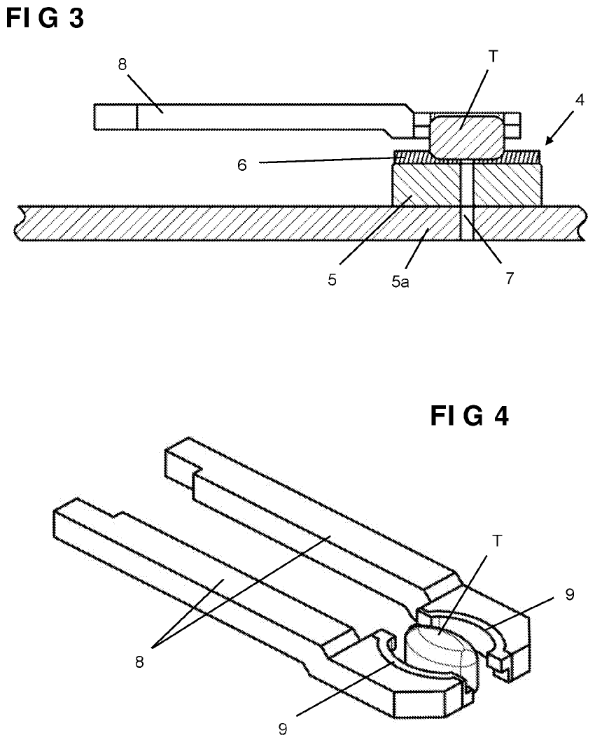

At least one of the examining stations 1 is a device for scanning or reflection measurement. Thus, for determining the active substance in a pharmaceutical product, measurements with NIR (Near Infra-Red), RAMAN and similar detectors are usual. In particular, for carrying out such scanning or reflection measurements as part of a test cycle for pharmaceutical products, this product, for example the tablet T shown in FIGS. 2 to 4, is taken by means of the robotic arm 2 to a tablet nest 4 (see FIGS. 2 and 3). This preferably consists of a solid, usually metallic lower part 5, if need be part of a carrier structure 5a, and, fastened thereon, a holder of elastically flexible material, in particular a plastic disk 6. A bore hole passes through the centre of the lower part 5 and the plastic disk 6, possibly protected by a window permeable to the radiation used, said bore hole constituting a fully coverable aperture for the radiation to the tablet T. The radiation used for the measurement can, for example, be supplied by way of a radiation guide 7. The lower section 5 and the plastic disk 6 on the other hand are radiation-impermeable. The robotic arm 2 can also be attached to the carrier structure 5a.

For all manipulating procedures the manner of gripping and clamping the products described below is advantageous. However, this applies in particular to the manipulation procedures in relation to the scanning and reflection measurement.

Preferably the tablet T is clamped in a form-fitting manner and thus securely held by means of two gripper jaws 8 of the holding mechanism 3 at the free of the robotic arm 2 that can be adjusted relative to each other. In accordance with an advantageous form of embodiment the gripper jaws 8 can also be sprung in order to compensate for the dimensional tolerances of the products or to react to changes in size during temperature changes of the products (change from warm to cold during tablet production). Due to this secure clamping, the pharmaceutical product can not only be placed down by the holding mechanism 3 of the robotic arm 2, but pressed onto the plastic disc 6 with an adjustable force so that form-fitting comes about between the lower side of the table T and the upper side of the plastic disk 6 which prevents any gaps for undesirable scattered light or scattered radiation in the direction of the radiation detector. For this, the transport system, in particular the robotic arm 2, is of course configured to exert a pressing force acting on the product in the direction of the support. In this way, at least for the scanning or reflection measurement the product T is fixed in place in relation to the device 1. This fixing is preferably achieved or supported in that a pressing force is exerted on the product in the direction of the support. In all events, according to the present invention, during at least part of the transport path the product is coupled with the transport system, preferably a holding mechanism 3 of the transport system configured as a robotic arm 2, in a friction-fitting and/or form-fitting manner.

However, fixing of the pharmaceutical product for the scanning or reflection measurement is not mandatory. Rather, it can be advantageous for certain measurements if the product is left movable during the scanning or reflection measurement. For example, through opening the gripper jaws 8 after placing down the product, a tablet T clamped in gripper jaws 8 is released for a short time and only for the scanning or reflection measurement. After completion of these examination procedures the product T is preferably coupled to the transport system again in the same way as described above through gripping with holding mechanism 3 thereof. Particularly on the movably left product T, but if need be also on products left coupled to the transport system, several measurements can advantageously be carried out, preferably at different points of the product T. Especially in the case of elongated or large-area products such as oblongs, measurements can be carried out at different points of the product and a mean value for the measurement over the entire product can be determined.

In order to improve the holding effects of the holding mechanism 3 on the robotic arm 2 on the pharmaceutical products even further, to make them more flexible and partially also to prevent the scattered radiation even more reliably, the following gripper jaw 8 variants in particular are possible in the context of the invention.

Thus the gripper surface facing the respective opposite gripper jaws 8 could be shaped so that for the product an at least two-point support for the product is formed. On the other hand the gripper surfaces of the gripper jaws 8 could also be shaped to complement at least part of the peripheral contour of the products. For very gentle clamping of the products in the gripper jaws 8 a form of embodiment can be advantageous in which the gripper surface of at least one of the gripper jaws 8 is covered with an elastic layer 9, preferably of a plastic material. This layer 9 can also constitute an additional seal to prevent undesirable scattered radiation during the scanning measurement.

In any event the holding mechanism 3 is designed in such a way that at least one of the gripper jaws 8 has at least one recess which is shaped so that for accommodating the pharmaceutical product between the contact surfaces there is an intermediate space, which can be enlarged to open the gripper jaw 8 device and made smaller to close it. The gripper jaws 8 are—possibly adapted or adaptable to the product—shaped in such a way that with the gripper jaws 8 open, the product to be tested can be introduced into the intermediate space via an access opening, and held in the intermediate space between the gripper jaws 8 in a form-fitting and/or friction-fitting manner when the gripper jaw 8 arrangement is closed. To do this the procedure is preferably as follows: the gripper jaws 8 of the holding mechanism 3 are opened, the product is then placed on a support surface, the holding mechanism 3 is preferably moved in such a way that the object comes to lie in the intermediate space. Finally the gripper jaws 8 are closed and through this the product is positioned and possibly orientated at a point determined by the shape of the contact surfaces. Preferably all the steps are carried out automatically, e.g. controlled by computer.

Furthermore, especially for exerting a pressing force directed towards the tablet nest 4 on the tablet T or every other pharmaceutical product on the gripper jaws 8, inwardly projecting protrusions, projecting beyond a clamped product T can be provided, which thereby slightly constrict the free opening above the product. The height of the gripper jaws 8 projecting beyond the base of the protrusion is preferably smaller than the thickness of the products.

The variant of the invention hitherto mentioned as an example used opposing pincer-shaped gripper jaws 8 which are closable on moving toward each other and openable when moving away from each other. These are preferably arranged in parallel to each other and are essentially movable transversely to their longitudinal extent, preferably held by a base unit of the holding mechanism 3 and by means of this holding mechanism 3 connected, for example, to the robotic arm 2. Particularly preferably the gripper jaws 8 are attached in the holding mechanism 3 in an interchangeable manner in order to adapt them to different types of pharmaceutical products T or for repair or replacement in the event of faults.

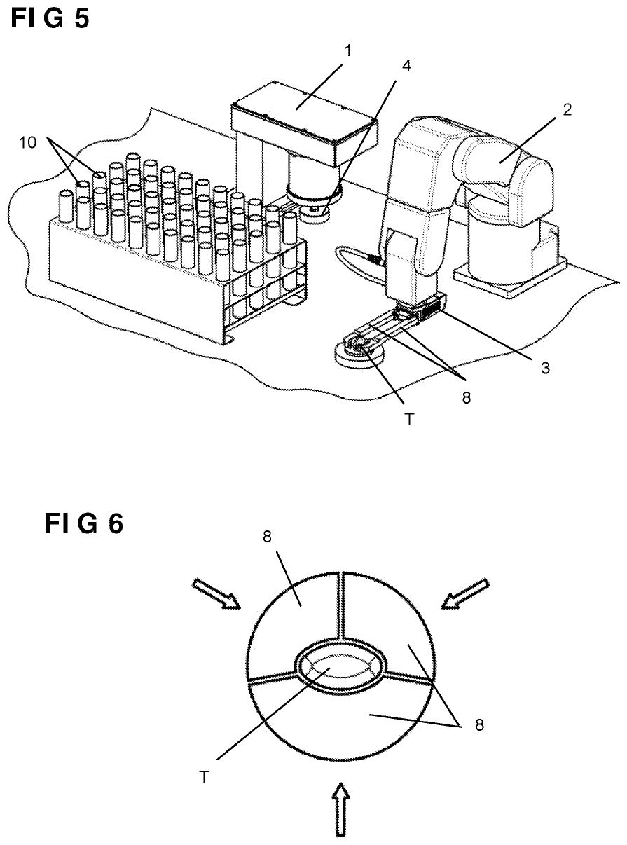

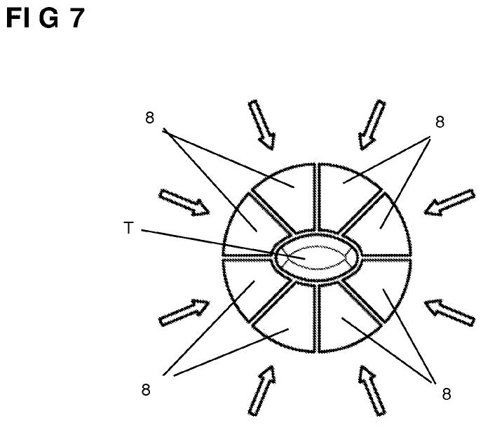

Alternatively, in addition to pincer grippers, which can, if need be, be supplemented with ejectors for any products T sticking to the gripper jaws 8, forms of embodiment are also possible in which the gripper jaws 8 are arranged along a closed contour. In FIGS. 6 and 7 such arrangements of gripper jaws 8 are shown, which can preferably completely surround and clamp the pharmaceutical product T along its periphery. They have a central free space for receiving the pharmaceutical product T and ensure a particularly picking up and holding effect on the pharmaceutical product T. Preferably the contours of the gripper jaws 8 facing the product T are shaped to be precisely complementary to the outer peripheral contour of the product T.

In the case of all gripper variants and forms of embodiment of gripper jaws 8 and transport system the controllable holding mechanism and/or at least one of the gripper jaws 8 can comprises sensors to determine the path of any gripper jaws 8 and/or the force exerted on the pharmaceutical products T. By way of a path sensor the working path travelled by the holding structure, for example at least one gripping jaw 8, during the holding of the product T, can be determined. In the control system of the holding mechanism 3 or of the entire transport system this current path is compared with a reference value which has previously been determined for a correct gripping procedure with a correct orientation of the pharmaceutical product T. Thus, not attaining the reference value can indicate an incorrect orientation of the product T, while exceeding it can be a sign of unintentional destruction of the product during the gripping procedure or also that there is no product present to transfer to the transport system 2, 3.

Similarly, via determination of the force required to grip the product and comparing this value with a reference value, conclusions about the presence and correct orientation of the product can be drawn. For forces greater than the reference value either incorrect gripper function with increased friction or jamming of the gripper jaws 8 can be concluded, or this indicates incorrect orientation of the project T. Force measurements below the reference value on the other hand can occur due the product not being present or an incorrect product (with less strength than expected).

Both procedures, alone or in combination, could of course subsequently prevent incorrect measurements due to incorrect positioning or the products or due to an incorrect product.

Should incorrect orientation of the product T be determined, a further embodiment of the device according to the invention with a turntable at the transfer position of the pharmaceutical product to the transport system can be used. On such a turntable, if incorrect orientation is detected, immediately before friction-fitting and/or form-fitting picking up by the transport system, the product T can be turned into the correct position for being taken over by the transport system.

Due to the free programmability of the robotic arm 2 and the fact that there are no predetermined paths for the pharmaceutical products due to transport system restrictions or path specifications, the examining stations of the device according to the invention can be travelled to in any sequence. After scanning and reflection measurement, this makes it possible to return the pharmaceutical product to an examination station 1 or a complete tablet testing device, in order, for example to measure the hardness or another physical parameter.

On the other hand, after the scanning or reflection measurement the tablets T or other products can also be placed directly into a tube 10 or other repository with freely programmable placement positions, as in FIG. 5. After the robotic arm reaches these positions, preferably with the middle of the product T within the edge of the tube 10, after opening of the gripper jaws 8 of the holding mechanism 2 the product automatically falls into the tube 10. However, after the completion of all desired tests, the product can also be transferred by means of the transport system to other examination or manipulation systems.

### LIST OF REFERENCE NUMBERS

1 Examining station
2 Robotic arm
3 Holding mechanism
4 Tablet nest
5 Lower part
5a Carrying structure
6 Plastic disk
7 Radiation guide
8 Gripper jaw
9 Elastic layer
10 Tablet tube
T Tablet

What is claimed is:

1. A device for determining at least one physical and/or chemical parameter of pharmaceutical products (T), such as tablets, pills, oblongs or similar, with at least one examining station (1) as well as with at least one automatic transport system for the products to and/or between and/or within and/or from one or more examining stations, wherein the transport system comprises a controllable holding mechanism (3) for a friction-fitting and/or a form-fitting holding of the pharmaceutical products (T), wherein the travel paths and spatial orientation of the holding mechanism (3) are freely programmable.

2. The device according to claim 1, wherein the controllable holding mechanism (3) is arranged on a freely programmable robotic arm (2).

3. The device according to claim 1, wherein at least one examining station (1) is a device for scanning or reflection measurement which comprises a support surface (5) for the pharmaceutical product (T).

4. The device according to claim 3, wherein the device for scanning measurement has a support (6) which is impervious to wavelengths used and, at least during the scanning measurement, the support holds the product at least partially in a form-fitting manner, the support comprising an aperture that can be fully covered by the product (T).

5. The device according to claim 4, wherein the support (6) is made of an elastically flexible material.

6. The device according to claim 1, wherein the holding mechanism (3) comprises at least two gripper jaws (8) that can be adjusted relative to each other.

7. The device according to claim 6, wherein the gripper surface of the respective opposite gripper jaws (8) is shaped in such that an at least two-point support is formed between the gripper jaws (8).

8. The device according to claim 6, wherein the gripper surfaces of the gripper jaws (8) are shaped to complement at least one part of a peripheral contour of the products (T).

9. The device according to claim 6, wherein the gripper jaws (8) are:
opposite each other in a pincer-like manner,
closable when moving toward each other, and
openable when moving away from each other.

10. The device according to claim 6, wherein the gripper jaws are:
arranged along a closed contour, and
have a central free space for accommodating the pharmaceutical product (T).

11. The device according to claim 6, wherein the controllable holding mechanism (3) and/or any gripper jaws (8) comprise sensors for determining the path of any gripper jaws (8) and/or the force exerted on the pharmaceutical products (T).

12. The device according to claim 1, wherein at a transfer position of the pharmaceutical product (T) to the transport system, a turntable is arranged on which the product (T) comes to lie immediately before being picked up in a friction-fitting or form-fitting manner by the transport system.

13. The device according to claim 6, wherein a gripper surface of at least one of the gripper jaws (8) is covered with an elastic layer (9) and/or a layer (9) which is impervious to wavelengths used in scanning measurements.

14. The device according to claim 6, wherein the gripper jaws (8) have protrusions projecting inwards beyond a clamped product (T) and wherein a height of the gripper jaws (8) projecting beyond the protrusion is smaller than a thickness of the products (T).

15. The device according to claim 4, wherein the transport system (2) is configured to exert a pressing force on the product (T) in a direction of the support (6).

16. A method of determining at least one physical parameter of pharmaceutical products (T), such as tablets, pills, oblongs and similar, wherein at least one chemical or physical property is examined and the product is transported and manipulated between and/or during and/or after at least one test procedure by an automatic transport system (2) wherein a transport path of the product (T) and its spatial orientation are pre-programmed at least partially independently of a spatial technical set-up and is travelled by means of a robotic arm (2).

17. The method according to claim 16, wherein at least one property of the product (T) is determined by means of a scanning and reflection measurement.

18. The method according to claim 17, wherein at least for the scanning or reflection measurement, the product (T) is fixed in place in relation to the device for scanning measurement, preferably with a pressing force being exerted in the direction of a support (6).

19. The method according to claim 17, wherein during the scanning or reflection measurement the product is left moveable, or wherein several measurements are carried out, preferably at different points of the products (T).

20. The method according to claim 16, wherein during at least part of the transport path, the product (T) is coupled to a holding mechanism (3) of the transport system in a friction-fitting and/or form-fitting manner.

21. The method according to claim 16, wherein after completion of all tests, the product (T) is deposited by the transport system (2) in a repository (10) or is transferred to other examining systems, repositories, or manipulation systems.
